(19) United States
(12) Patent Application Publication
(10) Pub. No.: US 2019/0057767 A1
(43) Pub. Date: Feb. 21, 2019

(54) SYSTEM, METHOD AND APPARATUS FOR DIAGNOSTIC ANALYSIS OF A MEDICAL IMAGING SYSTEM TO MAINTAIN COMPLIANCE, CONTINUITY AND REGULATORY GUIDELINES

(71) Applicant: Eric Michael Wilson, Fort Myers, FL (US)

(72) Inventor: Eric Michael Wilson, Fort Myers, FL (US)

(21) Appl. No.: 16/023,534

(22) Filed: Jun. 29, 2018

Related U.S. Application Data
(60) Provisional application No. 62/548,134, filed on Aug. 21, 2017.

(51) Int. Cl. G16H 30/20; G16H 40/20

(52) U.S. Cl. CPC G16H 30/20; G16H 40/20

(57) ABSTRACT

A system, method and apparatus for diagnostic analysis of a medical imaging system. The system is configured to extract and analyze header data for an imaging sequence that is available in digital imaging and communications in medicine (DICOM) standards to assist with the troubleshooting of diagnostic imaging systems and to improve image sequence compatibility between imaging systems and/or complying with regulatory bodies.

### CROSS-REFERENCE TO RELATED APPLICATION

This application claims the benefit of priority of U.S. provisional application No. 62/548,134, filed Aug. 21, 2017, the contents of which are herein incorporated by reference.

### BACKGROUND OF THE INVENTION

The present invention relates to all diagnostic imaging systems, and for reference I will use magnetic resonance image (MRI) scanners as an example because of the vast amount of parameters that can be altered by the end user.

Diagnostic imaging systems, such as MRI scanners, have many parameters that can be adjusted to effect image quality, such as contrast, time, signal-to-noise ratios, and the like. Over time, these adjustments may lead to maintenance or service intervention to determine why the clinical images of a patient do not look the same. Periodically all these imaging systems go through an accreditation process but are not clinically inspected other than the readings by the Radiologist and normal quality control.

Moreover, in larger hospital organizations that may have numerous MRI scanners, there is currently no method to compare image sequence parameters taken from one facility or imaging system to the other imaging systems within the organization.

Compatibility issues between multiple equipment vendors and their proprietary software present obstacles to troubleshooting and analysis. Most analytical software is done by proprietary systems maintained by each vendor, not by using a DICOM standard output to encompass all vendors' equipment.

As can be seen, there is a need for an improved system, method, and apparatus for the analysis of imaging systems, to improve clinical clarity, equipment availability, and system performance and maintain guidelines within a tolerance than can be altered to effect overall image quality.

### SUMMARY OF THE INVENTION

In one aspect of the present invention, a system for analysis of operational data of a medical imaging system is disclosed. The system comprising includes a medical imaging system and a digital imaging and communications in medicine (DICOM) compliant controller for the imaging system. The controller provides an image file including image data and header data. The header data specifies a plurality of operational performance parameters of the medical imaging system to produce the image data. A computer having a user interface with a program product comprising machine-readable program code for causing, when executed, the computer to perform process steps. The steps include extracting the header data from the image file and storing the header data in a data storage device.

In other embodiments, the steps include comparing one or more operational parameters of the header data to a reference parameter specified for the one or more operational parameters for the imaging system, A compliant header data record is stored in a compliant record database. A non-compliant header data record is stored in a non-compliant record database.

The system may also include a picture archiving and communications system (PACS) operatively connected to the DICOM compliant controller for storage of the image file. In this embodiment, the system entails extracting the header data from an image file stored on the PACS.

In other aspects of the system, a router is interposed between the DICOM compliant controller and the PACS. The router is configured to filter the image file to remove the image data prior to storage of the image file on the PACS.

The process steps may also include, separating patient identifying information from the header data and storing only the plurality of operational performance parameters of the imaging system.

Other process steps may include receiving the header data for a first diagnostic image; receiving the header data for a second diagnostic image, where the first diagnostic image and the second diagnostic image correspond to a same patient. The steps then compare one or more operational parameters of the first diagnostic image to the one or more operational parameters of the second image, to identify a difference in the one or more operational parameters. Alternatively, or in addition, the steps may include comparing one or more operational parameters of the first diagnostic image and the one or more operational parameters of the second image to a reference parameter specified for the one or more operational parameters for the imaging system.

In yet other implementations, the steps may include comparing the header data to an accredited header data, where the accredited header data contains operational performance parameters of the imaging system as certified by a regulatory body.

Other embodiments of the system include extracting a temporal data element from the header data for a specified image sequence for a plurality of image files; and analyzing the temporal data element to determine an operator performance metric.

These and other features, aspects and advantages of the present invention will become better understood with reference to the following drawings, description and claims.

### BRIEF DESCRIPTION OF THE DRAWINGS

FIG. 1 is a representative system architecture for diagnostic analysis of a medical imaging system.

FIG. 2 is an alternative system architecture for diagnostic analysis of a medical imaging system.

FIG. 3 is a flow chart for diagnostic analysis of a medical imaging system.

FIG. 4 is a diagram illustrating sample operational parameters for an imaging system.

FIG. 5 is a diagram of an alternative system architecture for diagnostic analysis of a medical imaging system data.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, embodiments of the present invention provides a system, method, and apparatus for analysis of data available in Digital Imaging and Communications in Medicine (DICOM) standards to assist troubleshooting of diagnostic imaging systems and to improve image sequence compatibility between devices, and/or over temporal spacing between image sequences. The diagnostic imaging system may be one or more of a magnetic resonance imaging (MR) or computerized tomography (CT) scan, and other DICOM compliant imaging systems.

Many hours of troubleshooting of diagnostic imaging systems result from the adjustment and calibration of system setting parameters being altered over time. Likewise, many OEM's for imaging systems spend millions of dollars per year in travel and services for follow up imaging system application visits.

An MRI Patient may be the subject of numerous imaging sequences (also called protocols) to complete a study. Due to physics involved with the imaging system there may be a give and take for image quality, time, contrast, signal to noise, and other parameters.

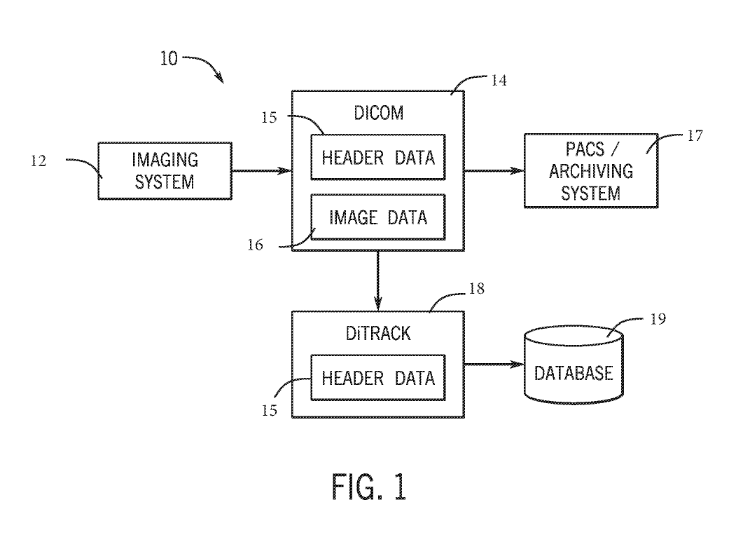

As seen in reference to FIG. 1 a system architecture for diagnostic analysis of a medical imaging system data is illustrated. In this embodiment, an imaging system 12 is operatively connected to a DICOM compliant controller 14 for the imaging system 12. The DICOM compliant controller 14 receives data associated with a digital image taken by the imaging system 12. The data includes header data 15 and image data 16. The header data 15 and image data 16 are typically stored on a picture archiving and communications system (PACS) 17. A DiTrack component 18, according to aspects of the invention, receives the header data 15 from the DICOM compliant controller 14. The received header data 15 may then be stored on a database associated with the DiTrack component 18.

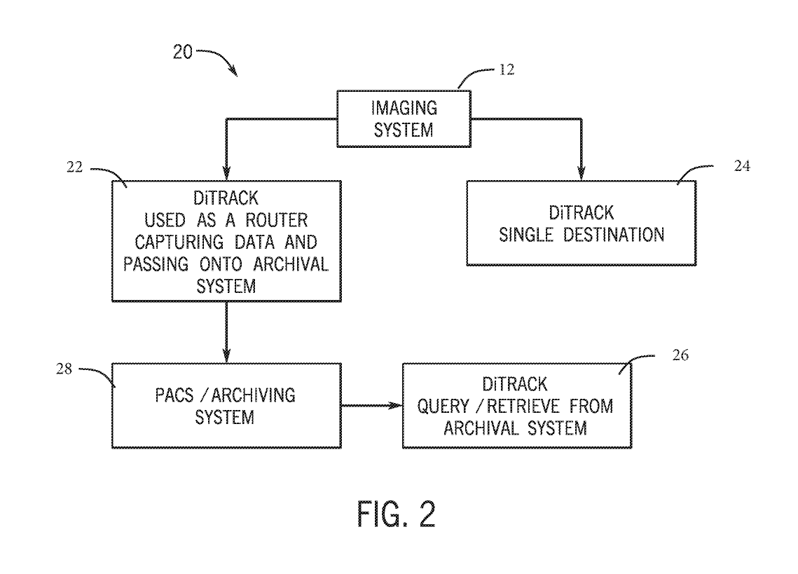

As seen in reference to FIG. 2, the DiTrack component 18 can be used as a variety of devices or modes of operation. In a first embodiment of device/mode 22 would be consistent with a router, or a filter that permits the passage of only certain data pertaining to image parameters of the imaging system 12. When used as a router or filter 22, the DiTrack component 18 is configured to filter and limit data passing through the filter to DICOM header data 15, which may then be utilized for analytical purpose, via other aspects of the diagnostics tracking (DiTracks) component 18 to in order to assess the performance of the imaging system 12 and its operation.

In another embodiment, the DiTrack Component 18 may be configured to exist as a DICOM destination 24, with the same intentions but is provided to receive and save only the DICOM header information 15. So this device a computer with software would be DICOM compliant among all vendors that transmit DICOM data 15.

In yet another embodiment, the DiTrack Component 18 can be configured as a plug in to a PACS 28. This would be a preferred implementation but would also require PACS vendors to implement such technology. In yet other embodiment, the DiTrack component 18 can be setup as a stand alone system 26 with access to a repository of DICOM header data 15.

Over time all patients that are captured on this system will be stored in a database 19 omitting image data 16, but storing all the Header Data 15. If the header data 15 includes patient identifying information, the header data 15 would need to be stored on a HIPAA compliant data storage 19. For applications of the present invention that do not require patient identifying information, patient identifying data may also be filtered from the DICOM header data 15 so that the system analysis header data 15 may be stored in a database 19 without the need for HIPAA compliant storage and safeguards.

There may be configuration differences depending upon whether the DiTrack system 18 is setup as a router 22 or as a stand-alone system 26. The DiTrack system 18 will be able to query the database 19 to compare and contrast different fields. One objective is to collect data and check for parameter consistency or variations, which over time can produce changes or similarities in the imaging sequence. This database 19 can be used for hospital centers with multiple imaging scanners 12. Alternatively, a single imaging system 12 can be analyzed to investigate image quality over time. Analysis of the header data 15 in database 19 may also provide a health system or vendor with the ability to determine if image quality variations are the result of system hardware deficiencies versus operator changes made to the imaging system's 12 parameters over time.

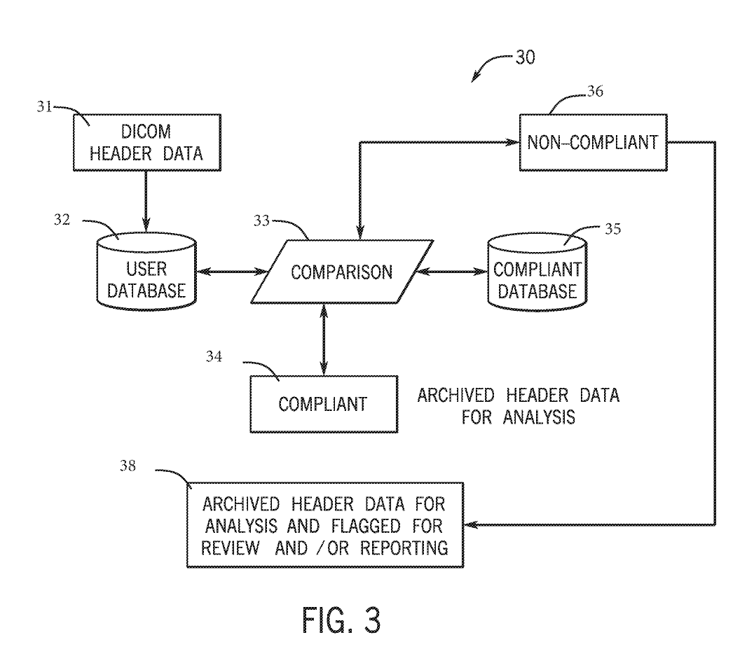

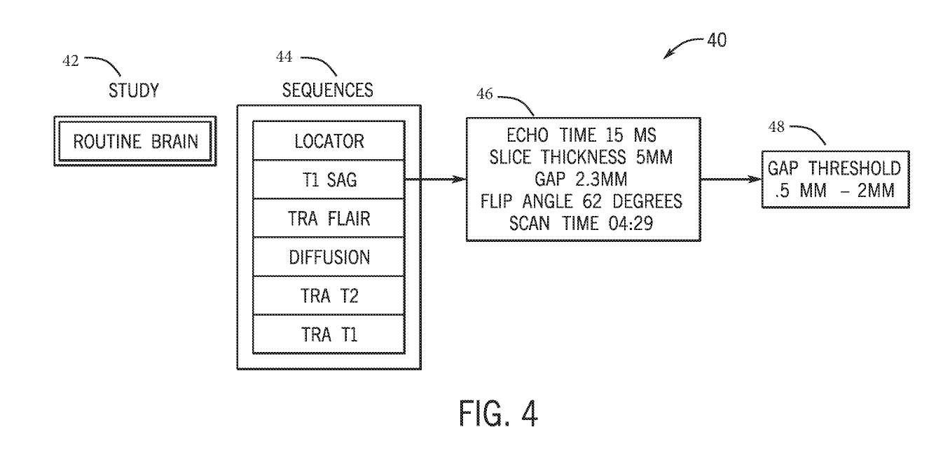

As seen in reference to FIGS. 3 and 4, the DiTrack system 18 includes a comparison module 30. The comparison module 30 receives DICOM header data 31 that has been provided to a user database 32. At block 33, the DICOM header data 31 is compared against configurable target, or reference parameters 46 based upon the use of the imaging system 12 and the DiTrack System 18.

By way of non-limiting example, shown in FIG. 4, the parameters 40 may relate to a study 42, such as a brain study. For each study 42, or target of an imaging, there may be one or more sequences 44, which may be specified in a study protocol, or specified by a requesting physician. The DICOM header information 46 for the selected T1 SAG sequence 44, includes a plurality of operational parameters that the imaging system 12 utilized to take a particular image. For example, an echo time, a slice thickness, a gap, a flip angle, and a scan time. In this instance, the operational parameters for the gap threshold, exceed the gap threshold parameter 48, and would be identified as non-compliant, while the parameters for echo time, slice, thickness, flip angle, or scan time, are identified as compliant.

If the DICOM header data for the record is identified as compliant, the data record may be tagged as compliant at 34 and archived in a compliant record database 35 for later analysis. At block 36, if one or more parameters of the DICOM Header data are identified as non-compliant, the data record is flagged for subsequent analysis and archived in a non-compliant database. The system 18 may be configured to provide an automatic notification for reporting purposes to one or more of the physician or maintenance personnel to determine the source of the discrepancy.

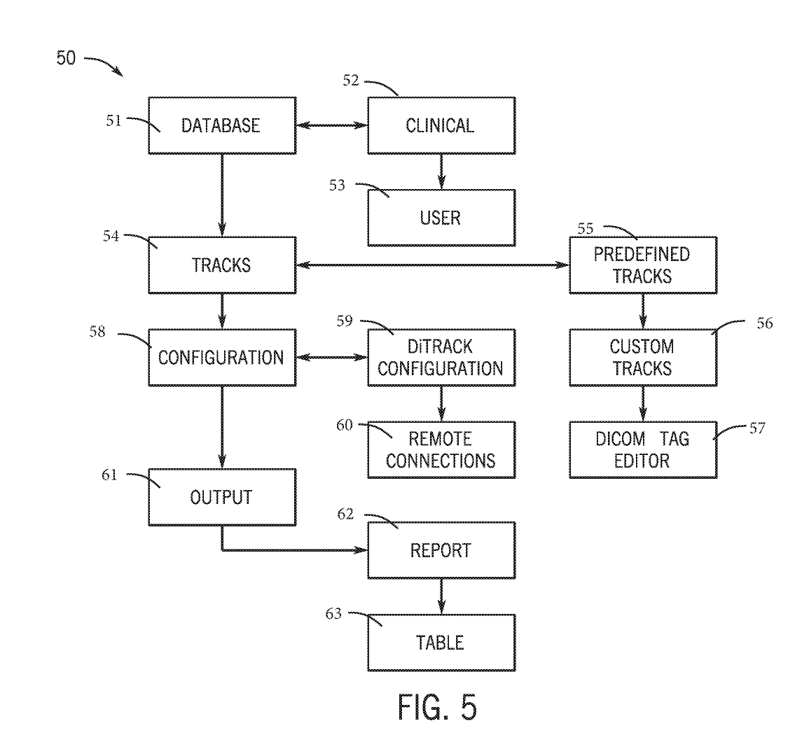

As seen in reference to FIG. 5, starting with a workstation, a diagnostic tracking software application (DiTrack) 18 is configured to accept DICOM header information 15. The DICOM header information 15 may be stored in a database 19. The DiTrack software application 18 may be configured to perform an analysis of the DICOM header information 15 in a plurality of diagnostic modules.

As seen in reference to FIG. 5, by way of non-limiting example, the diagnostic modules may include: clinical studies/compliance module, a single study quality control module; a multiple study quality control module; a multiple scanner comparison module; and a study comparison over time module. The various modules may compare and contrast DICOM data between: imaging sequences for different patients, different imaging systems; different operational parameters for the same imaging system, and imaging sequences for the same patient taken at different temporal periods. The parameters selected for study may be selected via a graphical user interface.

The DiTrack software which is the engine for the system, shows a basic table of logic relative to the software. Though this is the software in the basic state, it is customizable based on the specific application. The software will consist of a database 51. The system may be utilized on two modes both a clinical 52 and a user 53.

The clinical mode 52 is based on clinical compliance using thresholds both positive and negative to maintain continuity. The user mode 53 is based on customizable thresholds defined by the end user for their system needs that may vary from the compliance mode.

Moving through the software there are one or more tracks 54 which specify a compilation of DICOM header data to be used for the output, whether it be for a report 62 or a table 63 for review. The Track menu consists of one or more predefined tracks 55, one or more custom tracks 56, and a DICOM tag editor 57, The DICOM tag editor allows for input of explanations and definitions of all the DICOM tags available with a system. The compilation of predefined tracks 55 are basic sets that correspond to each of the four main usages of the DiTrack system. These four uses consist of a router 22, single destination 24, a query and retrieve 26 from an external system, and finally a PACS plugin 28. Furthermore the custom tracks 56 can be expanded on by the predefined tracks 56 or customizable tracks 56 by the end user for their specific application.

The DICOM Tag Editor 57 the system will house a definition database of all the local and OEM defined DICOM tags. Progressing to the configuration 58 are local software configurations 59 and remote connections 60. Local software configurations 59 include a GUI setup, a Networking setup, and Report 62 as well as Table 63 configurations for system outputs.

The Remote Connections 60 refer to one or more networking nodes that are being configured as destinations for sending and receiving DICOM data. Finally the Output 61 of the system is designed for both reports 62 and tables 63. Tables 63 are spreadsheet type tables to view outputs of both in and out of compliance as well as user determined tables for specific needs. The Reports 62 may be generated for compliance as well as user information that needs to be flagged for inconsistencies and/or review.

### EXAMPLES

Scenario 1.) In this example, a patient has a routine head MRI imaging sequence taken in 2012. In 2015, the same patient gets scanned again on the same imaging system 12 and the image quality is not comparable. The question presented, is whether the disparity in image quality is due to a scanner hardware defect or whether the system parameters are consistent with the scan performed in 2012.

In this case, the DICOM header information of the first scan and the second scan may be extracted and the operational parameters 46 for the first and second scans compared to the reference parameters 48 to identify any discrepancies from the reference parameters 48. In addition, the operational parameters 48 for the first and second scans may be compared to one another to identify differences in the operational parameters 48 between the first and second scans. In this case, it may be appropriate for an OEM to have a DiTrack system 18 installed onsite to avoid costs associated with application service visits.

Scenario 2)

In this example, hospital X has 13 MRI scanners 12 throughout their organization. The radiologists are concerned about image quality throughout the organization. The organization also desires to assess quality control issues, in terms of throughput and consistency. For example, does one site take 30 minutes to perform a knee imaging sequence, while another site only takes 20 minutes. The organization also wants to determine whether the imaging sequences are the same across sites and whether the image quality is consistent across sites. With reductions in reimbursements, facilities are trying to maximize throughput while maintaining consistency and compliance.

In this instance, the DiTrack system 18 allows the organization to retrieve and compare the operational parameters from the DICOM header data to provide a comparison of the multiple scans against the reference parameters. Variances in the operational parameters may be used to identify a failing imaging system 18 or may be used to identify inconsistencies between operators among the facilities. These inconsistencies may identify facility or individual procedural or training deficiencies for correction.

In addition to the operational parameters of the imaging system 18, the DiTrack system 18 also permits the organization to make assessments according to temporal data extracted from the DICOM header data. As will be appreciated from Applicant's disclosure, the DiTrack system 18 may also be configured to provide performance data analytics, such as number of patients scanned per day, per facility, to determine patient throughput tied to employee productivity.

Scenario 3)

In this example, a clinical drug study takes place on multiple scanners 12 over time. Though the patient information may be archived according to Health Level Seven (HL7) international standard on the hospital network, the diagnostic imaging information is typically stored on a PACS 17. The diagnostic imaging information data stored on PACS 17 are not currently used for compliance and continuity assessment.

In this scenario, the DICOM header data 15 may be sanitized to remove patient identifying data and may be maintained separately from the HL7 compliant data. The DiTrack system 18, may then be utilized to assess the imaging systems 12 to show consistencies and/or inaccuracies that may be relevant to the study. This could provide other than the Radiologist reading to limit the exams that fall out of compliance.

Scenario 4)

In this scenario, a regulatory body accredits an imaging system 12. Though not scheduled to look at clinical images for roughly two years, acquiring and holding the header data 15 for comparison to an accredited header data 15′ can be accessed and looked up at any given time between clinical acceptances. The ability to compare ongoing header data 15 with the accredited header data 15′ provides a check and balance system for assessing clinical quality against an accredited set point over time.

The system of the present invention may include at least one computer with a user interface. The computer may include any computer including, but not limited to, a desktop, laptop, and smart device, such as, a tablet and smart phone. The computer includes a program product including a machine-readable program code for causing, when executed, the computer to perform steps. The program product may include software which may either be loaded onto the computer or accessed by the computer. The loaded software may include an application on a smart device. The software may be accessed by the computer using a web browser. The computer may access the software via the web browser using the internet, extranet, intranet, host server, internet cloud and the like.

The computer-based data processing system and method described above is for purposes of example only, and may be implemented in any type of computer system or programming or processing environment, or in a computer program, alone or in conjunction with hardware. The present invention may also be implemented in software stored on a non-transitory computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a wide range of industries. To the extent the present application discloses a system, the method implemented by that system, as well as software stored on a computer-readable medium and executed as a computer program to perform the method on a general purpose or special purpose computer, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the invention as set forth in the following claims.

What is claimed is:

1. A system for analysis of operational data of a medical imaging system, the system comprising:
a medical imaging system;
a digital imaging and communications in medicine (DICOM) compliant controller for the imaging system, the controller providing an image file including image data and header data, the header data specifying a plurality of operational performance parameters of the imaging system to produce the image data;
a computer having a user interface; and
a program product comprising machine-readable program code for causing, when executed, the computer to perform the following process steps:
extracting the header data from the image file; and
storing the header data in a data storage device.

2. The system of claim 1, further comprising:
comparing one or more operational parameters of the header data to a reference parameter specified for the one or more operational parameters for the imaging system; and
storing a compliant header data record in a compliant record database.

3. The system of claim 2 further comprising:
storing a non-compliant header data record in a non-compliant record database.

4. The system of claim 1, further comprising:
a picture archiving and communications system (PACS) operatively connected to the DICOM compliant controller for storage of the image file.

5. The system of claim 4, wherein the header data is extracted from an image file stored on the PACS.

6. The system of claim 4, further comprising:
a router interposed between the DICOM compliant controller and the PACS, the router configured to filter the image file to remove the image data prior to storage of the image file on the PACS.

7. The system of claim 1, further comprising:
separating patient identifying information from the header data; and
storing only the plurality of operational performance parameters of the imaging system.

8. The system of claim 1, further comprising:
receiving the header data for a first diagnostic image;
receiving the header data for a second diagnostic image, wherein the first diagnostic image and the second diagnostic image correspond to a same patient;
comparing one or more operational parameters of the first diagnostic image to the one or more operational parameters of the second image, to identify a difference in the one or more operational parameters.

9. The system of claim 1, further comprising:
receiving the header data for a first diagnostic image;
receiving the header data for a second diagnostic image, wherein the first diagnostic image and the second diagnostic image correspond to a same patient.
comparing one or more operational parameters of the first diagnostic image and the one or more operational parameters of the second image, to a reference parameter specified for the one or more operational parameters for the imaging system;

10. The system of claim 1, further comprising:
comparing the header data to an accredited header data, the accredited header data containing operational performance parameters of the imaging system as certified by a regulatory body; and
determining a source of variance between the header data and the accredited header data.

11. The system of claim 1, further comprising:
extracting a temporal data element from the header data for a specified image sequence for a plurality of image files; and
analyzing the temporal data element to determine an operator performance metric.
